(19) United States
(12) Patent Application Publication
(10) Pub. No.: US 2021/0237060 A1
(43) Pub. Date: Aug. 5, 2021

(54) PAPER-BASED THREE-DIMENSIONAL STRUCTURE MICROCHIP FOR DETECTING TARGET ANTIGEN BY USING IMMUNOCHEMICAL ASSAY, AND METHOD FOR DETECTING TARGET ANTIGEN BY USING SAME

(71) Applicant: UNIANCE GENE CO.,LTD, Seoul (KR)

(72) Inventors: Byungkeun OH, Seoul (KR); Kwanwoo SHIN, Seoul (KR); Myeongjun LEE, Seoul (KR); Soum VEASNA, Seoul (KR); Youngkee KIM, Gwangju (KR); Sangnam LEE, Seoul (KR)

(21) Appl. No.: 17/050,621

(22) PCT Filed: Apr. 11, 2019
(86) PCT No.: PCT/KR2019/004344

§ 371 Date: Oct. 26, 2020

(30) Foreign Application Priority Data
Apr. 27, 2018 (KR) 10-2018-0048965

(51) Int. Cl. B01L 3/00; G01N 33/558

(52) U.S. Cl. CPC B01L 3/502715; B01L 2300/0825; G01N 33/558

(57) ABSTRACT

An example microfluidic device includes: paper; a pattern film; a conjugate pad and absorption pad, which are inserted into holes formed in the pattern film and come into contact with the paper, are inserted in separately formed holes, respectively, and the absorption pad is placed at a fixed interval between the holes, and an aptamer and antibodies combined with the sensing material are stored in above a conjugate pad; a reaction pad with an antibody that is located over a conjugate pad and absorption pad above and uniquely binding to antigen contained in the specimen; and a cover film attached to the pattern film; and a space formed by removal of the film at the bottom of the reaction pad.

### TECHNICAL FIELD

This invention is about a microfluidic device of the paper based three-dimensional structure for detecting the target antigen using the immuno-chemical diagnostic method, and the method of detecting the target antigen using it, in more detail, the method of controlling the speed and direction of the fluid, and the method of detecting the target antigen using the three-dimensional structure of the microfluidic device in a single specimen injection without external power.

### BACKGROUND ART

ELISA (Enzyme-linked immunosorbent assay) is one of the most widely used immunoassay methods, which detects target proteins present in specimens, and the detection of target proteins is made possible by antigen-antibody reactions. ELISA is based on the principle that antibodies or antigens are absorbed (immunosorbent) on solids and can be divided into direct ELISA, indirect ELISA and sandwich ELISA depending on how antibodies are utilized.

Although ELISA is used a lot because it has high precision and reliability, it takes a long time to measure. Meanwhile, sensor-based lap-on-a-chip had a problem with short reaction time and automatic reaction, while external fluid flow devices were required and difficult to handle.

Recently, a paper stripe kit with gold nanoparticle base with ease of use and simplified measurement steps was proposed (Fu et al. 2017, Zhao et al, 2008). However, the paper stripe kit of the gold nanoparticle base presented in the paper had the limitation showing a low sensitivity depending on the probe.

To increase probe sensitivity, the use of probes using chromogenic enzyme, such as HRP, or luminous materials, was proposed. However, there was still a problem with adding steps to the substrate to remove unresponsive enzymes or specimen solutions to increase accuracy (sensitivity) of discoloration. To solve this problem, there is a way to remove the substrate after the immune response and then drop the enzyme on the new substrate, but there is a limit to commercial performance as humans have to remove the substrate.

As such, paper-based devices are now being used as bio-sensors because they are cheap, light, and flexible. However, paper-based devices use only wet force of paper, so not only is the speed of movement low, but it is also difficult to control direction such as speed control and jumping to other channels. As such, paper-based devices had limitations in number of reactions, reaction rate, and directional control, resulting in longer measurement times, especially had a limitation on automatic implementation of complex immunochemical reactions.

### SUMMARY OF DISCLOSURE

### Technical Problem

This invention provides paper-based microfluidic device for immunochemistry diagnosis that can control speed and direction.

This invention provides microfluidic device that can automatically perform complex immune chemical reactions within paper chips without external power.

This invention provides paper-based microfluidic device that dramatically reduce the time it takes to diagnose diseases.

This invention provides paper-based microfluidic device that can detect various diseases and viruses.

This invention provides paper-based microfluidic device with high sensitivity and high selectivity for specific antigens.

### Technical Solution

A microfluidic device for target antigen detection by Enzyme linked immunosorbent assay, comprising,

paper (10)

a pattern film (20) attached to above paper and formed microtubule a pattern;

a conjugate pad (30) and absorption pad (40), which are inserted into the holes formed in above a pattern film and come into contact with above paper, are inserted in separately formed holes, respectively, and absorption pad (30) is placed at a fixed interval between the holes, and an aptamer and antibodies (32) combined with the sensing material (31) are stored in above a conjugate pad (30);

a reaction pad (50) with an antibody (51) that is located over a conjugate pad and absorption pad above and uniquely binding to antigen (1) contained in the specimen;

a cover film (60) attached to a pattern film (20) above, and

a space (A) formed by removal of the film (20) at the bottom of the reaction pad (50),

Wherein specimen, cleaning solution and substrate solution are provided to each microtubule patterned part, the specimen and cleaning solution are moved to the conjugate pad, the reaction pad and an absorption pad, and the substrate solution is automatically supplied to the space (A) below the reaction pad

A microfluidic device for target antigen detection by Enzyme linked immunosorbent assay, comprising

Paper (10)

A pattern film (20) attached to the paper above, containing 1st hole (21), where absorption pad is inserted and the response pad is placed, the 2nd hole (22) located apart from the 1st hole above, on which absorption pad is inserted where reaction pad is located above of it, and a microtubule patterns (23) in which the specimen, cleaning solution and substrate solution move by capillary force;

a conjugate pad (30) where antibody (32) or aptamer, inserted into the 1st hole above and combined with the sensing material (31);

an absorption pad (40) inserted in the second hole above and sucking in specimen, cleaning solution and substrate solution;

a reaction pad (50) secured with antibody (51) which is located over above a conjugate pad and absorption pad connecting together, uniquely binding with antigen (1) contained in above-mentioned specimen on the bottom side; and

a cover film (60) attached to the pattern film (20) above,

wherein the 1st and the 2nd holes and the microtubule patterns are perforated and specimen, cleaning solution and substrate solution move along the microtubule pattern on the paper,

wherein the microtubule patterns (233) of substrate solution is connected with a second hole to move the substrate solution into a space (A) below the reaction pad.

A method for target antigen detection using a microfluidic device by Enzyme linked immunosorbent assay, comprising the steps of

providing specimen, cleaning solution, and substrate solution to each microtubule a patterned part formed on paper;

moving the specimen and the cleaning solution to a conjugate pad, a reaction pad and an absorbing Pad in sequential order through above microtubule patterned part with no external power source;

moving the substrate solution to a space (A) under above reaction pad by capillary force without external power sources through above microtubule patterned part,

when the specimen reaches above conjugate pad, wherein the method makes antigen (4) contained in the specimen and the sensing antibody (32) of a conjugate pad or aptamer antigen/antibody react with antigen antibody and move it to above response pad,

when specimen containing antigen-sensing antibody and unreacted sensing antibody reach the reaction pad, wherein the method makes antigen-sensing antibody and unreacted sensing antibody antigen/antibody react with antibody fixed in the bottom of above reaction pad,

when above substrate solution reaches the reaction pad, wherein the method make enzyme reaction between the substrate and the sensing antibody or the sensing material bound to aptamer.

### Advantageous Effects

Through the speed and direction control of fluid and the three-dimensional structure of chips, this invention can provide microfluidic device of paper-based three-dimensional structure that can detect target antigens with a single injection of specimen without external power.

This invention can increase the speed of movement as the fluid moves along a patterned film microtubule (fine flow channel), unlike the conventional method in which the fluid is absorbed and moved inside the paper. In addition, this invention can control fluid velocity and direction through hydrophilic surface treatment and width and length control of microtubule on paper under the microtubule.

This invention prevents specimen or cleaning solution from seeping into the substrate solution path (substantive solution microtubule) because the path through which the substrate solution flows and the path through which the specimen and the cleaning solution flow are located on different planes (3D structure, bridge structure). In other words, the 3D microfluidic device structure of this invention can reduce signal noise and waste of substrate solution by preventing enzyme reactions in areas other than reaction pad due to leakage of specimen or cleaning solution.

### BRIEF DESCRIPTION OF DRAWINGS

FIG. 1 is the full perspective view of one example of this invention.

FIG. 2 is the exploded view of FIG. 1.

FIGS. 3a, 3b and 3c illustrate an assembly process of FIG. 1.

FIG. 4 is a sectional view of a-a′ from FIG. 2.

FIG. 5 shows that leakage is prevented by a 3D structure (bridge structure).

FIGS. 6a and 6b are conceptual diagrams that show the prevention of leakage through the barrier area in the bridge structure.

FIG. 7 shows the measurements of the speed on paper based on coating and channel width.

FIG. 8 shows controlling the direction of capillary flow through speed control.

FIGS. 9a, 9b, 9c, 9d and 9e illustrate the method of detecting the target antigen of this invention.

FIG. 10 is the full perspective view of a different implementation example of this invention.

FIG. 11 is an exploded view of FIG. 10.

FIGS. 12a, 12b and 12c illustrate an assembly process of FIG. 10.

FIG. 13 is a section of the 3D (bridge) structure of FIG. 10.

FIG. 14 shows the discoloration of 3D microfluidic device and reaction pads according to antigen concentration of this invention manufactured in Example 1.

FIGS. 15a and 15b are graphs showing signal strength by adjusting antigen concentration of a specimen.

FIG. 16 shows the measurements of the signal strength of 10 nM Trx, PSA, HSA and BSA.

FIG. 17 shows the discoloration of 3D microfluidic device and reaction pad according to antigen concentration of this invention manufactured in Example 2.

FIG. 18 is the graph showing the measurements of the signal strength by adjusting antigen concentration of the specimen in Example 2.

### DETAILED DESCRIPTION—BEST MODE

In the following, the desirable working manner of this invention is illustrated by drawings. However, the scope of this invention is not limited to the description or drawings of the following manner. In other words, the terms used in this Specifications were used only to illustrate specific examples of implementation, not to limit this invention. The expression of singularity includes multiple expressions, unless the context clearly implies otherwise. In addition, the terms “include” or “have” as described in this Specifications shall be understood to specify that the features, numbers, steps, actions, components, components or combinations of them exist, and not to exclude in advance the existence or addition of one or more other features or numbers, steps, actions, components, components, or any combination of them.

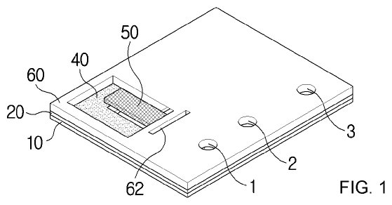

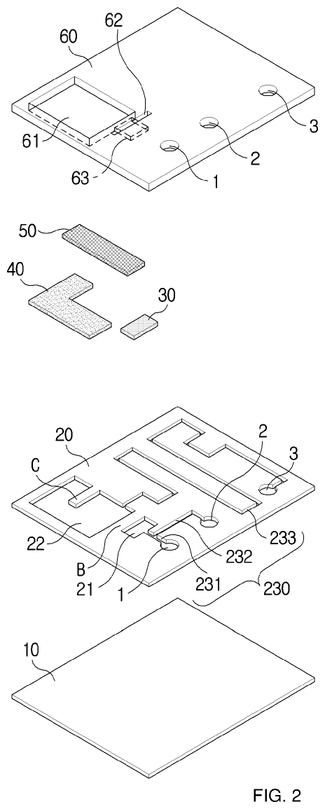

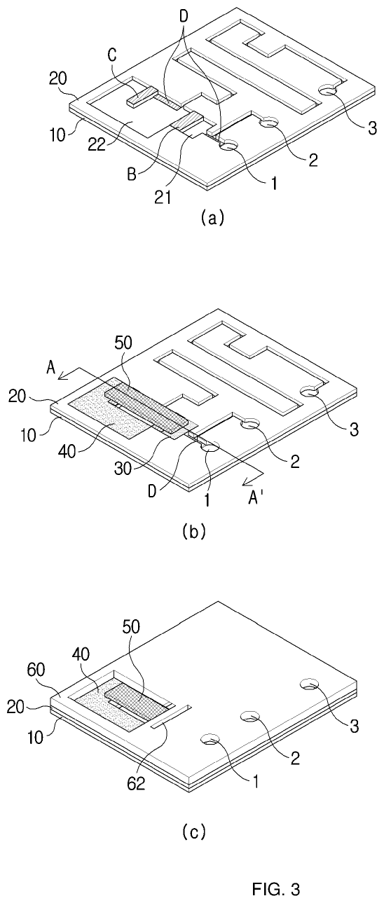

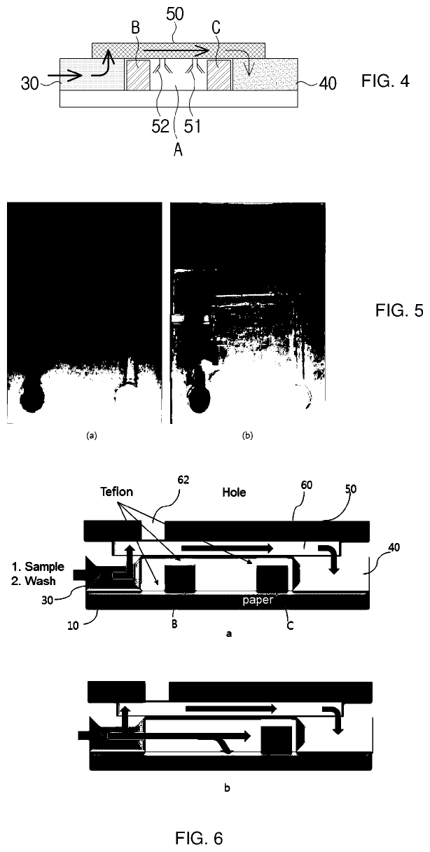

FIG. 1 is the full perspective view of the one example of this invention, FIG. 2 is the exploded view of FIG. 1, FIG. 3 illustrates assembly process of FIG. 1, and FIG. 4 is the sectional view of A-A′ from FIG. 3.

Referring to FIGS. 1 through 4, the paper-based three-dimensional structure of this invention includes paper (10), a pattern film (20), a conjugate pad (30), an absorption pad (40), a reaction pad and a cover film (60).

The paper (10) is the base layer of microfluidic device, providing capillary force to the fluid. Above paper is used as the more hydrophilic compared to above film. Above paper may be used unlimitedly for paper-based microfluidic device or for publicly notified paper used in sensors. For example, the paper above may be a paper or photographic paper used in Whatman Chromatography.

There is no special limit on the thickness of the paper above. For example, the thickness of above paper may be between 100 and 10000 μm.

The pattern film (20) above is a film attached to the paper above and formed with microtubule patterns (23). A pattern film above may be a publicly acknowledged plastic film, for example, PET or PE film. Above a pattern film is not limited in thickness. As an example, the thickness of a pattern film may be 50 μm to 500 μm.

Referring to FIGS. 2 and 3, the pattern film (20) includes the first hole (21) in which a conjugate pad (30) is inserted, the second hole (22) in which absorption pad is inserted and the specimen, cleaning solution and substrate solution move by the capillary force.

The microtubule patterns (23) include the specimen microtubule pattern (part) (231), the cleaning solution microtubule pattern (part)(232), and the substrate solution microtubule pattern (part) (233).

A pattern film (20) above may contain a specimen injection part (1), a cleaning solution injection part (2), and a substrate solution injection part (3).

The upper and lower sections of the 1st and 2nd holes and the microtubule patterns are perforated.

This invention can form the microtubule channels (they can be fine fluid channels) by attaching the pattern film (20) on the paper (10) above. In other words, above paper forms bottom parts of the microtubule channels while simultaneously providing the power source of the fluid flow, and above microtubule patterns form the side wall of the microtubule channels so that the fluid can travel through the microtubule patterns on the paper. When the fluid spreads throughout the paper and permeates the paper, the speed of movement was very slow and it was virtually difficult to move the fluid in the desired direction, but this invention can move the fluid in the desired direction quickly because it can be moved on the paper through microtubule channels formed on the paper.

The conjugate pad (30) and the absorption pad (40) are inserted in separate and formed holes, respectively, and each side is positioned at a prescribed interval between each other, and each side is in contact with above paper.

More specifically, the conjugate pad (30) above may be inserted in the first hole (21) above and stored or attached to one or more of the sensing antibodies (32), aptamer, or target antigen sensing complex with a sensing material (31) inside or at the top.

In the text below, the sensing antibody means an antibody (32) in which the sensing substance (31) is combined, but in the text below, it may be used as a term for inclusion of an aptamer or a complex for detecting target antigen.

The conjugate pad may contain (storage) sensing materials and use fibers that can be taken out under certain conditions, for example, glass fiber membrane.

Antibody above refers to a protein that is uniquely combined to antigen contained in the specimen to produce an agglutination reaction. Antibody used in the invention is not particularly specific, and any class of antibodies, if specifically combined with antigens, is possible. Also, although it is not specifically limited to antibodies that originate from animal species, antibodies from rabbits, goats and mice are desirable because they are relatively easy to acquire and have many examples of use.

Above aptamer may be a single-stack nucleic acid (DNA, RNA, or modified nucleic acid) with a stable trigeminal structure on its own and characterized by high affinity and specificity to the target molecule.

Above target antigen detection complex may be a composite containing nanoparticles and antibodies, and more specifically, a combination of nanoparticles, aptamers and antibodies.

For above target antigen detection complex, see Patent No. 10-1613020 of this applicant. Note that above target antigen detection complex includes nanoparticles, aptamers which is attached to the surface of above nanoparticles, and that are uniquely bound to antibody's Fc (crystallizable fragment) area, and the multiple number of antibodies of which Fab (antibody binding fragment) area lies in the opposite direction to above aptamers, while above aptamer is amplified by a real-time polymerase chain reaction (RT-PCR) to detect amount of target antigens bound to above antibody.

Above nanoparticles may be gold, silver, or silica. Above nanoparticles may be in sizes of 10 to 100 nm. If above nanoparticles are gold, S (sulfur) of above aptamer may be combined with above gold nanoparticles.

Above sensing materials may be substances capable of coloring, fluorescent, luminous, or infrared reactions in response to certain substances (e.g., substrate).

For example, above sensing materials may be nanoparticles such as enzymes, enzyme balls, gold nanoparticles, gold-enzyme complex particles.

For example, above enzymes include, but are not limited to, enzymes that catalyze color, fluorescence, luminescence, or infrared reactions, and, for example, alkaline phosphatase, β-galactosidase, horseradish peroxidase (HRP), luciferase and cytochrome P450.

Above enzyme balls may include enzymes and antibodies. For example, above enzyme balls may contain enzymes, albumin compounds, and antibodies. For above enzyme balls, Patent No. 10-1622477 of this applicant may be referred. For reference, above enzyme balls include antibodies attached to the surface of above albumin agglutinate (particle) and albumin agglutinate (particle) that contain above multiple enzymes by forming multiple enzymes, self-aggregates, above multiple number of enzymes exist dispersed in albumin agglutinate, and above-mentioned multiple number of enzymes are capable of enzyme reactions with multiple number of substrates penetrating inside albumin agglutinate.

Above enzyme is contained in above albumin nano agglutinate by 1 to 30 percent weight, above albumin is a bovine serum albumin, human serum albumin or a fragment of thereof, and above albumin agglutinate may be 100 to 300 nm in size.

Above absorption pad (40) is inserted in the second hole (22) above and sucks up specimen, cleaning solution and substrate solution. Above absorption pad (40) can provide power for fluid to travel by capillary force. For above absorption pad, a good absorbent fiber, for example, cellulose membrane, polyester, polypropylene and glass fiber, can be used.

Above reaction pad (50) is located across above-mentioned the conjugate pad and the absorption pad and connects them, and is secured with an antibody (51) that is uniquely coupled to antigen (1) contained in above specimen, and the antibody (52) for control, respectively on the lower surface of the reaction pad.

The reaction pads of this invention may be nitrocellulose membrane with excellent absorption and protein adhesion, polyfluorinated Vinylidene (PVDF), etc.

Referring to FIGS. 2 through 4, above reaction pad (50) is located in a different plane from above-mentioned a conjugate pad and absorption pad, so space (A) can be formed between above reaction pad and the paper located below it.

In this invention, the structure of the conjugate pad, the absorption pad, and the reaction Pad, which form the lower space (A) in the reaction pad (50), is described as bridge or 3D structure. This bridge or 3D structure is the general concept of a structure in which the path through which the substrate solution flows and the path through which the specimen and cleaning solution flow are located on different planes, so that these paths do not physically contact each other. In other words, a conjugate pad (30), the reaction pad (50), and absorption pad (40) are physically in contact with each other to move specimen and cleaning fluid through absorption force of the membrane or capillary force, but the path of the substrate solution is physically separated from these three pads, preventing the specimen or cleaning solution from entering absorption or capillary force into the substrate solution pathway.

In particular, the reaction pad of this invention has excellent absorption, so specimen and cleaning solution do not penetrate into the pad and flow into space (A).

The substrate solution can be injected into above space (A) through above substrate solution microtubule pattern (233) to contact the reaction pad located at the top of above space (A).

FIG. 5 shows that leakage is prevented by a 3D structure (bridge structure). FIG. 5a is the 3D (bridge) structure of this invention, and 5b is the plane structure in which the substrate solution is injected into the path side of the reaction pad. In case of FIG. 5b, red ink flows into the substrate solution injection path, but in case of 5a, it can be confirmed that no leakage occurs in the substrate solution path. In the case of 5b, the substrate solution reacts with a specimen having a coloring enzyme in the substrate solution microtubule before reaching the reaction pad, generating signal noise, resulting in wasted substrate.

Also, the microfluidic device of this invention may contain a barrier area (B) that prevents the specimen solution or cleaning solution from leaking into the substrate solution micro-channel after entering space (A). Above barrier area (B) is the film layer that remains unremoved between the first and second holes above in A pattern Film (20).

In addition, the upper part of the barrier area (B) above may be coated with hydrophobic substance (e). There are no special restrictions on above hydrophobic substances. For example, above hydrophobic substance may be Teflon.

FIG. 6 is a conceptual diagram that shows the prevention of leakage through the barrier area in the bridge structure. FIG. 6b does not have barrier B, so the specimen solution passed through a conjugate pad (30) can be moved to space (A) through paper (10), but 6a shows that the coating of barrier B and hydrophobic material prevents the specimen from moving to space (A).

If the specimen, cleaning solution, and substrate solution are provided in each microtubule infusion part (1, 2, 3), the microfluidic device in FIG. 1˜4 can move the specimen and cleaning solution sequentially through capillary force and above microtubule without external power sources to above conjugate pad, above reaction pad and absorption pad. In the microfluidic device above, when above cleaning solution is moved to above absorption pad, above substrate solution can be automatically supplied to the space (A) below the reaction pad through capillary force and microtubule of above substrate solution without external power source.

Above specimen microtubule pattern part (231) and the cleaning solution microtubule pattern part (232) may be connected to the first hole after being combined into a pattern channel, or may be connected to the first hole, respectively.

As shown in FIG. 3a, above paper located at the bottom of above specimen microtubule pattern part (231) is coated with hydrophilic material to provide a faster flow speed than above cleaning solution microtubule pattern part or substrate solution microtubule pattern part. Above hydrophilic material may be a hydrophilic polymer or a hydrophilic metal. For example, silver may be coated with above hydrophilic material.

In addition, above microfluidic device may contain a hydrophilic substance (D) coated on paper forming the bottom of above space (A). Above hydrophilic material (D) allows the substrate solution to move rapidly throughout the reaction pad in above space (A).

Above specimen microtubule pattern part (231) has a wider a pattern width than above cleaning solution microtubule pattern part or substrate solution microtubule pattern part, which can provide fast flow rate.

Above substrate solution microtubule pattern part (233) is narrower, has longer a pattern length than above specimen microtubule pattern part (231) and above cleaning solution microtubule pattern part (232), and not coated with hydrophilic material, allowing the substrate solution to reach the second hole later than the specimen or cleaning solution.

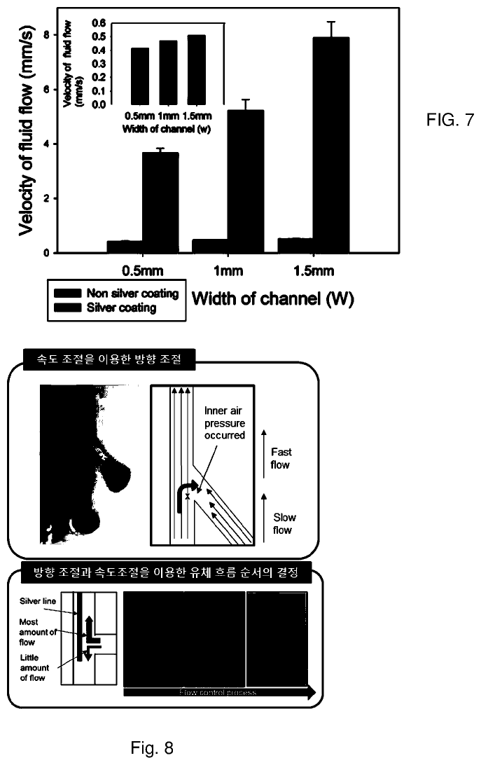

FIG. 7 shows the measurements of the speed on paper according to coating and channel width. If you refer to FIG. 7, when the channel size width is 0.5 mm, without silver coating, the speed is 0.41 mm/s, when the channel size width is 1.5 mm, and if silver is coated, the speed is 7.90 mm/s, showing that the channel width can be tripled and nearly 20 times faster in silver coating.

FIG. 8 shows the direction of capillary flow is controlled by speed control. In FIG. 8, if fluid is spilled into the main channel with narrow channel width and silver coated and to the branch channel with a narrow width which enters to the main channel, by capillary force, the fast fluid attempts to enter the branch channel of the slow fluid. However, a slow fluid forms an air wall at the intersection, and an air wall not only prevents the fast fluid from entering the branch channel but also prevents the slow fluid from entering the main channel. As shown in FIG. 8, after all the fluid supplied to the main channel is flown through, the fluid in the branch channel can enter the main channel.

As such, the microfluidic device of this invention can move specimen, cleaning solution and substrate solution sequentially to above reaction pad without external power source by speed control of fluid flowing through microtubule.

The cover film (60) above may inhibit the flow of above specimen on the top surface of the reaction pad. The cover film above prevents specimen overflowing or entering a conjugate pad and entering the substrate solution microtubule pattern.

The cover film (60) contains a perforated measuring hole (61) to allow exposure of part of the reaction pad located in the lower part. The signal reader can calculate antigen concentration by reading the light or color change of the exposed reaction pad through above measuring hole (61).

A cover film (60) above may contain a hole (62) which reduces capillary force near the inlet of the reaction pad from which the specimen and specimen are introduced.

On the underside of above response pad, a sink dent (63) may be formed, and a reaction pad may be inserted into above sink dent.

A cover film above includes holes (1, 2, 3) drilled in the same positions as the specimen injection part (1), the cleaning solution injection part (2) and the substrate solution injection part (3) of a pattern film above.

In another respect, this invention presents a method of detecting target antigen using a microfluidic device of paper-based three-dimensional structure. FIG. 9 indicates the method of detecting the target antigen.

The method of detecting target antigen in this invention includes the step of providing specimen, cleaning solution and substrate solution, the step of transferring specimen and cleaning solution to the conjugate pad, the reaction pad, and the step of transferring substrate solution to the lower part of the reaction pad.

The specimen above include antigen (4).

The solution available for use in ELISA antigen-antibody reaction notified for above cleaning solution may be used without restriction.

For above substrate, a substance that reacts uniquely with above enzyme may be used. For example, horse radish peroxidase is used as the enzyme, chloronaphthol, aminoethylcavazole, diaminobenzidine, D-Luciferin, Lucigenin (vis-N-methylacrydinium nitrate), Lesorupine benzyl ether, luminol, amplex red reagent (10-acetyl-3, 7-dihydroxyphenoxazin), TMB(3,3,5,5-tetramethylbenzidine), ABTS (2,2′-Azine-di [3-ethylbenzthiazoline sulfonate]) or o-phenylendiamine (OPD) may be used,

when alkaline phosphatase is used as the enzyme above, bromoclophosphate (BCIP), nitro-blue tetrazoleum (NBT), naphthol-AS1-B1-phosphate (naphthol-AS-B1-phosphate) or ECF (enhomiflorus) may be used as the substrate.

In this invention, specimen, cleaning solution, and substrate solution are dropped into the specimen injection parts (1, 2, 3) of the 3D structure. In this invention, the three solutions are dropped on the microfluidic device at the same time and ELISA reaction can be automatically carried out.

As mentioned above, specimen and cleaning solution are moved through the microtubule (231, 232) above to a conjugate pad (30), reaction pad (50), and absorption pad (40) sequentially without external power source.

Referring to FIG. 9, in this invention, when the specimen reaches above a conjugate pad (30), antigen (4) contained in the specimen and antibody (32) or aptamer, or the target antigen detection complex (hereinafter referred to as the sensing antibody (32)) are antigen/antibody reacted, and then move to above reaction pad (9b).

If the specimen containing antigen (4)-sensing antibody (32) and unreacted sensing antibody reach above reaction pad (50), antigen-sensing antibody and unreacted sensing antibody shall be fixed by antigen/antibody-reacting to antibody (51) (9c).

In above method, when above specimen and cleaning solution are absorbed in absorption pad after passing through the reaction pad, above substrate solution is injected into the space (A) below above reaction pad through above microtubule (233). When above substrate solution reaches the reaction pad, an enzyme reaction is performed between the sensing antibody (31) and the substrate (9d, 9e).

Above method may include step to produce antigen concentrations by reading the signal from the enzyme reaction. A commonly known device or method for measuring color change or luminescence intensity resulting from an enzyme reaction may be used without restriction.

As noted above, above method may be used to separate a conjugate pad from above absorption pad at prescribed intervals and place over above paper and to locate above reaction pad over above a conjugate pad and above absorption pad to form a space (A) where substrate solution enters between above response pad and the paper.

In addition, above method can control the speed and direction of specimen, cleaning solution, and substrate solution by coating the top side of the paper located at the bottom of the microtubule with hydrophilic material or adjusting the width of the microtubule.

The method of detecting target antigen using microfluidic device of three-dimensional structure in this invention can be referred to the details of microfluidic device of paper-based three-dimensional structure described above.

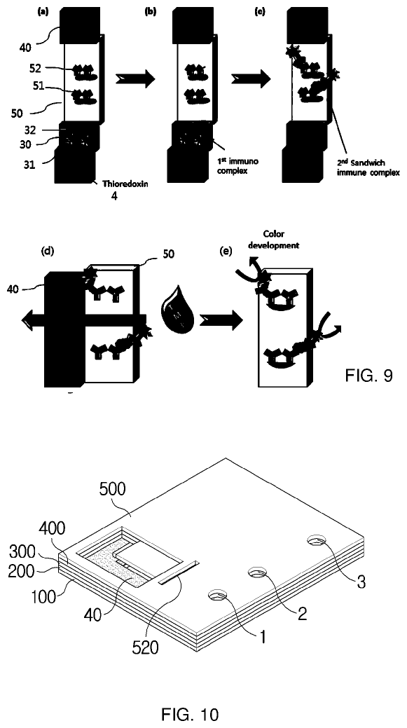

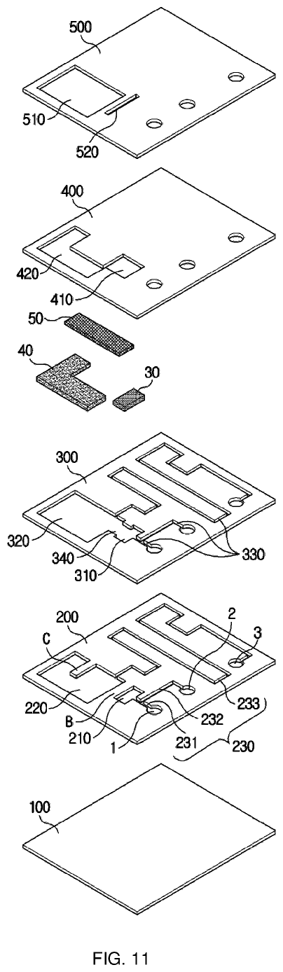

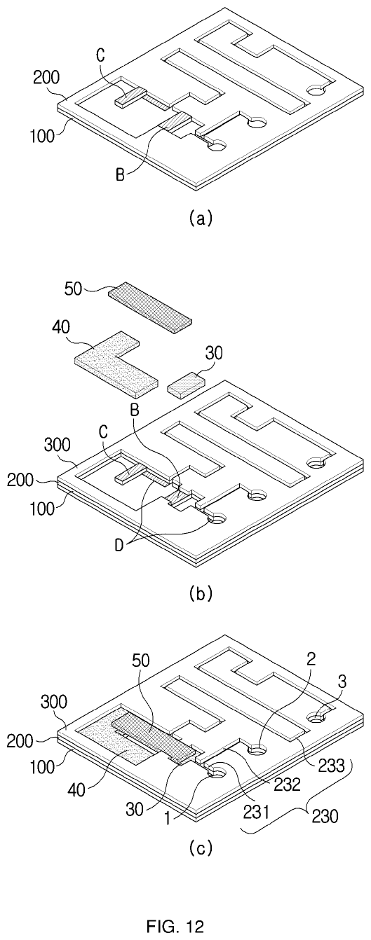

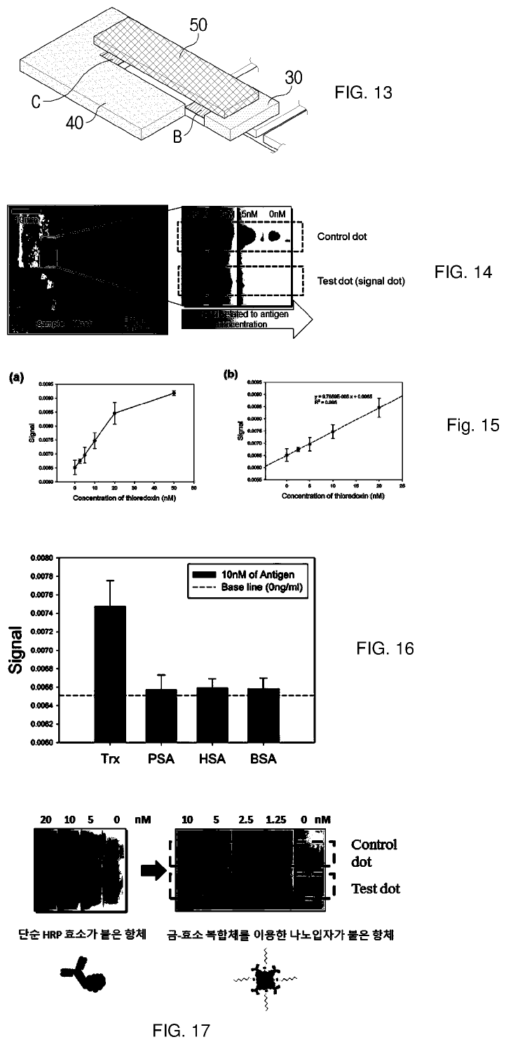

FIGS. 10 to 13 indicate different implementation examples of this invention. FIG. 10 is the full perspective view of the other implementation example of this invention, FIG. 11 is the exploded view of FIG. 10, FIG. 12 shows assembly process of FIG. 10, and FIG. 13 is the cross section of bridge structure of FIG. 10. FIGS. 10 to 13 show a microfluidic device structure that partially modifies the structure of FIGS. 1 to 4.

Referring to FIGS. 10 to 13, the microfluidic device of this invention includes paper (100), first a pattern film (200), second a pattern film (300), a conjugate pad (30), absorption pad (40), reaction pad (50), third a pattern film (400), and a pattern 4 film (500).

For paper (100), a conjugate pad (30), absorption pad (40), and Response Pad (50), see FIGS. 1 to 4.

The first a pattern film (200) is attached to the paper above and includes the 1st hole (210), where a conjugate pad is inserted, the 2nd hole (220), which is separated from the 1st hole above, where absorption pad is inserted and the response pad is located, and the microtubule pattern part (230), in which the specimen, cleaning solution and substrate solution move by capillary force.

Above microtubule pattern part (230) includes the specimen microtubule pattern part (231), the cleaning solution microtubule pattern part (232), and the substrate solution microtubule pattern part (233).

A pattern film (200) above may contain specimen injection part (1), cleaning solution injection part (2), and substrate solution injection part (3).

A pattern film (200) above includes film area B between the 1st hole (210) and the 2nd hole (220). As mentioned above, film area B may form a barrier area of 3D structure.

Above hole 220 may contain a boundary area (C) on the film where absorption pad, which is a “ [symbol] ” shape, can be inserted and secured.

Referring to FIG. 12a, above first a pattern film (200) may be synthesized on top of the paper (100) and then coated with hydrophobic material on film B and C.

See also 12a, the 1st a pattern film (200) above may be joined on the paper (100) above and then coated with hydrophilic material (D) on the lower paper of the specimen microtubule pattern section (231) and on the paper at the entrance of the substrate solution of space (A).

The second a pattern film (300) has the same a pattern as the first a pattern film (200) above, but a third hole (340) is formed to further strain the barrier area (B) of the film to secure the reaction pad.

Referring to FIG. 12, the second a pattern film is joined on top of the first a pattern film and the conjugate pad, absorption pad and reaction pad are inserted into the first hole (210, 310), the second hole (220, 320), and the third hole (340), respectively.

The third a pattern film (400) above may contain the 4th hole (410) formed to expose the 1st and 3rd holes above and the 5th hole (420) formed to expose absorption pad above.

The third a pattern film (400) above may be transparent.

Meanwhile, on the microfluidic device in this invention, the fourth a pattern film (500) can be attached to the second a pattern film above without the third a pattern film (400).

Above fourth a pattern film (500) may include the 6th hole (510) formed to expose the 2nd hole and the ceiling hole (520) formed to expose the entrance side of above reaction pad to the direct lower part. Above fourth a pattern film may be referred to a cover film described above.

The 6th hole (510) above may correspond to the measuring hole (61) of a cover film above.

Above substrate solution microtubule pattern part (233) is connected to the second hole to move the substrate solution into the space (A) below above reaction pad.

Above paper located in the lower part of a pattern part of above specimen microtubule (231) is coated with hydrophilic material to provide a faster flow rate compared to a pattern part of the cleaning solution microtubule or substrate solution microtubule.

Above specimen microtubule pattern part (231) has a wider a pattern width than above cleaning solution microtubule pattern part or substrate solution microtubule pattern part, which can provide fast flow rate.

Above substrate solution microtubule pattern part (233) may reach the second hole later than the specimen or cleaning solution because a pattern width is wider or longer than above specimen microtubule pattern part (231) and above cleaning solution microtubule pattern part (232).

### DETAILED DESCRIPTION—MODE FOR INVENTION

Below, this invention is described in detail by reference to the implementation examples and drawings attached. However, attached examples illustrate the specific implementations of this invention and do not intend to limit the scope of the invention's rights.

### Implementation Example 1

Paper and each film layer are produced in the structure shown in FIGS. 10-˜13. Cricut explore Air 2′ (Provo Craft & Novelty, Inc) was used for cutting. For paper, an optical paper was used, and for film, 100 [symbol] PET film was used.

After attaching the film 1 (200) to the paper, a silver coating (D) was applied to the paper on the top of the microtubule 231 and to the paper on the entrance side of the hole. I put silver in a 0.5 mm pen and drew a line on the paper with a pen, and dried it at 60° C. for about 20 minutes.

The film 2(300) was put together at the top of the film 1.

A conjugate pad solution was prepared with 0.5% BSA solution 1 μl, 40% trehalose 2.5 μl, 0.01% Tween 20.6 μl, and HRP combined Anti-trx antibody 0.12 μl. The mixture was dropped into a 7 mm×4 mm glass fiber membrane and dried at room temperature for about four hours.

1 [symbol] (0.2 mg/ml) Rabbit Anti-trx antibody was dropped onto the nitro cellulose membrane (10 um pore size; 4 mm×25 mm) on one side of the reaction pad to form a test dot (size 1 mm). A control dot was made by dropping Rabbit anti-Mouse IgG antibody 1 [symbol] (0.1 mg/ml) on the nitro cellulose membrane (reaction pad). It was cleaned with a 0.1% tween solution and dried. 0.5% BSA solution was dropped onto nitrocellulose membrane, filled the pore, and cleaned. It was dried at room temperature for an hour.

The absorption pad is a cellulose membrane in an “ [symbol] ” shape bent shape.

The conjugate pad, the absorption pad and the reaction pad were inserted into each hole of the second a pattern film and the first a pattern film. Again, the third a pattern film and the fourth a pattern film were stacked sequentially on top of the second a pattern film in order to manufacture a microfluidic device.

The size of the microfluidic device is 55 mm×45 mm and weighs 1.5 g. One chip has a silver-coated specimen solution channel (1 mm×8 mm), not silver-coated cleaning channel (1 mm×20 mm), and substrate solution channel (0.5 mm×210 mm). If three solutions (see Table 1 below, 10 μl specimen solution, 20 μl cleaning solution, 100 μl TMB substrate solution) are simultaneously deposited and waited for about 12 minutes, all solutions flow sequentially without external force and react automatically. After the substrate reaction, the signal is generated as shown in FIG. 3.

Table 1 below shows specimen, cleaning and substrate solution content, volume and the time these solutions reach the reaction pad through each microtubule.

TABLE 1

| | | Time to reach | |
|---|---|---|---|
| Solution | Content | reaction pad | Volume |
| Specimen | 0.05% BSA PBS solution + Trx | 3 sec ± 1 sec | 10 [symbol] |
| Cleaning | 0.1% Tween 20 PBS(V/V) | 25 sec ± 5 sec | 20 [symbol] |
| Substrate | 3,3,5,5-tetramethylbenzidine(TMB) | 8 sec ± 30 sec | 150 [symbol] |

### Implementation Example 2

Except for the manufacture of a conjugate pad solution as follows, the same procedure was performed as the Implementation example 1.

First, an antibody with HRP attached to 0.5 ml of gold particles was mixed with 10 ul of 0.2 mg/ml and reacted for 15 minutes, followed by adding 10 ul of 0.5 mg/ml of thiolate polyethylene glycol, and then this mixture was left to have a reaction for 10 minutes. After centrifugation of the reactant at 13,000 RPM for 20 minutes, the top liquid was removed, it was cleaned with 0.01% Tween PBS, and stored with 0.05% BSA 200 ul and 0.05% Tween solution 200 ul. a conjugate pad solution was manufactured with 24 μl of completed gold-enzyme nanoparticles and 2 [symbol] of 40% trehalose. The mixture was dropped into a 7 mm×4 mm glass fiber membrane and dried at room temperature for about four hours.

FIG. 14 shows the discoloration of 3D microfluidic device and reaction pad according to antigen concentration of this invention manufactured in the Implementation example 1. FIG. 15 is a graph showing the measurements of signal strength by adjusting antigen concentration of a specimen. Signals were analyzed with the non-color image analysis program BIO-VALUE. The Trx concentration is inversely proportional to the RGB mean, and in FIG. 15 the Y axis is 1/(RGB mean).

Referring to the FIG. 15, it shows linearity between the Trx and the signal strength at 0 to 60 nM, especially at 0 to 20 nM.

FIG. 16 shows the measurements of the signal strength of 10 nM Trx, PSA, HSA and BSA. Each solution was prepared with 260 ng/ml of PSA solution, 660 ng/ml of BSA solution and 665 ng/ml of HSA solution.

The baseline (dotted line) in FIG. 16 is the signal strength measured in absence of an antigen. The base line signal strength is 0.006511. Compared to this base line, the signal strength increased little despite addition of PSA, BSA and HSA solutions. On the other hand, Trx was 0.0075, a significant increase in signal strength. Meanwhile, when Trx is 2.5 nM, the signal strength was 0.006742, which is stronger than the other three antigens of 10 nM. In other words, the device in this invention shows a high level of selectivity for Trx.

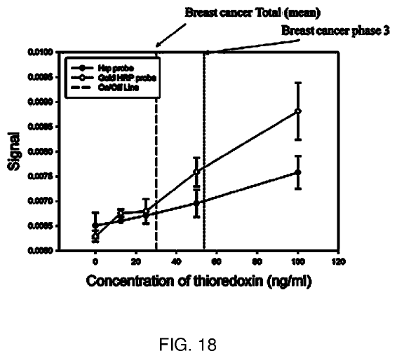

FIG. 17 shows the discoloration of the 3D microfluidic device and reaction pad according to antigen concentration in this invention manufactured in Example 2. FIG. 18 is a graph that shows the measurements of signal strength by adjusting antigen concentration of specimen. Referring to FIG. 18, the use of gold-enzyme complex nanoparticles produces stronger signals than conventional HRP-only sensing antibody, and also shows better sensitivity.

In above, the desirable implementation examples of this invention have been described in detail, but they are only for the purpose of explanation, not limiting the scope of protection of this invention.

This invention can be used as a microfluidic device for diagnosis of immunochemistry that can control speed and direction.

1. A microfluidic device for target antigen detection by Enzyme linked immunosorbent assay, comprising;
Paper (10)
a pattern film (20) attached to above paper and formed microtubule a pattern;
a conjugate pad (30) and absorption pad (40), which are inserted into the holes formed in above a pattern film and come into contact with above paper, are inserted in separately formed holes, respectively, and absorption pad (30) is placed at a fixed interval between the holes, and an aptamer and antibodies (32) combined with the sensing material (31) are stored in above a conjugate pad (30);
a reaction pad (50) with an antibody (51) that is located over a conjugate pad and absorption pad above and uniquely binding to antigen (1) contained in the specimen;
a cover film (60) attached to a pattern film (20) above, and
a space (A) formed by removal of the film (20) at the bottom of the reaction pad (50),
Wherein specimen, cleaning solution and substrate solution are provided to each microtubule patterned part, the specimen and cleaning solution are moved to the conjugate pad, the reaction pad and an absorption pad, and the substrate solution is automatically supplied to the space (A) below the reaction pad.

2. A microfluidic device for target antigen detection by Enzyme linked immunosorbent assay, comprising
Paper (10)
A pattern film (20) attached to the paper above, containing 1st hole (21), where absorption pad is inserted and the response pad is placed, the 2nd hole (22) located apart from the 1st hole above, on which absorption pad is inserted where reaction pad is located above of it, and a microtubule patterns (23) in which the specimen, cleaning solution and substrate solution move by capillary force;
a conjugate pad (30) where antibody (32) or aptamer, inserted into the 1st hole above and combined with the sensing material (31);
an absorption pad (40) inserted in the second hole above and sucking in specimen, cleaning solution and substrate solution;
a reaction pad (50) secured with antibody (51) which is located over above a conjugate pad and absorption pad connecting together, uniquely binding with antigen (1) contained in above-mentioned specimen on the bottom side; and
a cover film (60) attached to the pattern film (20) above,
wherein the 1st and the 2nd holes and the microtubule patterns are perforated and specimen, cleaning solution and substrate solution move along the microtubule pattern on the paper,
wherein the microtubule patterns (233) of substrate solution is connected with a second hole to move the substrate solution into a space (A) below the reaction pad.

3. The microfluidic device of claim 1, wherein the specimen, cleaning solution, and substrate solution are provided in each microtubule pattern, the specimen and cleaning solution sequentially move through the microtubule patterns by capillary force to the above conjugate pad, the above reaction pad and an absorption pad without external power source,
Wherein the cleaning solution move to an absorption pad, the substrate solution move to the space (A) under the reaction pad by capillary force through above microtubule patterns without external power sources.

4. The microfluidic device of claim 1, when the specimen reaches the conjugate pad, the microfluidic device moves the specimen to the reaction pad after the sensing antibodies or the aptamer in the conjugate pad make antigen-antibody reaction with the antigen (4) contained in the above specimen,
when specimen containing antigen-sensing antibody and unreacted sensing antibody reach the reaction pad, the microfluidic device above makes antigen-sensing antibody and unreacted sensing antibody react with antibody (51) and fix,
The microfluidic device above is a paper-based three-dimensional structure microfluidic device characterized by enzyme reaction between the substrate and the sensing material (31) combined with the sensing antibody when the substrate solution reaches the reaction pad.

5. The microfluidic device of claim 1, wherein the film area (B) between the first and second holes form a barrier layer that prevents above specimen and cleaning solution from leaking into above space (A).

6. The microfluidic device of claim 5, wherein the upper part of above film area (B) is coated with hydrophobic material.

7. The microfluidic device of claim 1, wherein the cover film (60) contains a hole (61) that reduces capillary force near the entrance of the reaction pad in which the specimen is introduced.

8. The microfluidic device of claim 1, wherein the paper is higher hydrophilicity compared to the films.

9. The microfluidic device of claim 1, wherein the specimen microtubule pattern part (213) and the cleaning solution microtubule pattern part are combined into one a pattern channel and then connected with the first hole.

10. The microfluidic device of claim 1, wherein the specimen microtubule pattern part (231) and the cleaning solution microtubule pattern part (232) are connected to the first hole.

11. The microfluidic device of claim 9, wherein the paper located in the lower part of above specimen microtubule pattern part (231) is coated with hydrophilic material to provide a faster flow rate than above cleaning solution microtubule pattern or substrate solution microtubule a pattern.

12. The microfluidic device of claim 9, wherein the specimen microtubule pattern section (231) has a wider a pattern width than above cleaning solution microtubule pattern part or substrate solution microtubule pattern part, which features faster flow rate.

13. The microfluidic device of claim 1, wherein the substrate solution reaches the 2nd hole later compared to the specimen solution or cleaning solution, since above substrate solution microtubule pattern has a wider or longer a pattern width than above specimen microtubule pattern part (231) and above cleaning solution a pattern part (232).

14. The microfluidic device of claim 1, wherein the reaction pad (50) is used with a cellulose and a polyvinyl-based membrane to move specimen or cleaning solution along the inside or lower surface.

15. A microfluidic device for target antigen detection by Enzyme linked immunosorbent assay, comprising;
Paper (100)
the first a pattern film (200) that contains the first hole (210), where a conjugate pad is inserted, the second hole (220), located apart from the 1st hole above, on which absorption pad is inserted and the microtubule pattern part where specimen, cleaning solution and substrate solution move by capillary force (230);
the second a pattern film (300) in which the same a pattern as the first a pattern film (200) is formed, but the film area (B) between the first hole (210) and the second hole (220) is additionally formed to secure the reaction pad (240);
a conjugate pad (30) in which a sensing antibody (32) inserted in the first hole above and combined with the sensing material (31) or aptamer is stored;
an absorption pad (40) inserted in the second hole above and sucking in specimen, cleaning solution and substrate solution;
the reaction pad (50) where antibody (51) is fixed that is located over above-mentioned a conjugate pad and absorption pad and connects them and is uniquely bound to antigen (1) contained in above-mentioned specimen.
the third a pattern film (400) containing the fourth hole formed to expose the first and third holes above, and the fifth hole formed to expose absorption pad above;
includes the fourth a pattern film (500) containing a 6th hole (510) formed to expose the 2nd hole above and a ceiling hole (61) formed to expose the entrance side of the reaction pad above at the direct lower side,
a microfluidic device of paper-based three-dimensional structure characterized by that the holes and microtubule patterns are perforated up and down, and specimen, cleaning solution, and substrate solution move along above microtubule patterns on the paper,
a pattern part of the substrate solution microtubule (233) above is connected with a second hole to move the substrate solution into the space (A) below the reaction pad above.

16. The microfluidic device of claim 15, wherein the film area (B) between the first and second holes forms a barrier layer that prevents above specimen and cleaning solution from leaking into above space (A).

17. The microfluidic device of claim 16, wherein the upper part of above film area (B) is coated with hydrophobic material.

18. The microfluidic device of claim 15, wherein the paper located at the bottom of above specimen microtubule pattern part (231) is coated with hydrophilic material to provide a faster flow rate than above cleaning solution microtubule pattern or substrate solution microtubule a pattern.

19. The microfluidic device of claim 15, wherein the specimen microtubule pattern part (231) provides fast flow velocity due to its wider a pattern width compared to above cleaning solution microtubule pattern part or substrate solution microtubule pattern part.

20. The microfluidic device of claim 15, wherein the substrate solution reaches the 2nd hole later compared to the specimen solution or cleaning solution, since above substrate solution microtubule pattern has a wider or longer a pattern width than above specimen microtubule pattern part (231) and above cleaning solution a pattern part (232).

21. A method for target antigen detection using a microfluidic device by Enzyme linked immunosorbent assay, comprising the steps of
providing specimen, cleaning solution, and substrate solution to each microtubule a patterned part formed on paper;
moving the specimen and the cleaning solution to a conjugate pad, a reaction pad and an absorbing Pad in sequential order through above microtubule patterned part with no external power source;
moving the substrate solution to a space (A) under above reaction pad by capillary force without external power sources through above microtubule patterned part,
when the specimen reaches above conjugate pad, wherein the method makes antigen (4) contained in the specimen and the sensing antibody (32) of a conjugate pad or aptamer antigen/antibody react with antigen antibody and move it to above response pad,
when specimen containing antigen-sensing antibody and unreacted sensing antibody reach the reaction pad, wherein the method makes antigen-sensing antibody and unreacted sensing antibody antigen/antibody react with antibody fixed in the bottom of above reaction pad,
when above substrate solution reaches the reaction pad, wherein the method make enzyme reaction between the substrate and the sensing antibody or the sensing material bound to aptamer.

22. The method of claim 21, the method separates a conjugate pad from above absorption pad at a prescribed interval and place it on above paper,
wherein above reaction pad is placed over above a conjugate pad and above absorption pad to form a space (A) where substrate solution flows between above reaction pad and the paper.

23. The method of claim 21, wherein the speed and direction of the specimen, the cleaning solution and the substrate solution is controlled by coating the top of paper located at the bottom of microtubule with hydrophilic materials or adjusting the width of microtubule.

24. The method of claim 21, wherein the method comprising the steps of, calculating antigen concentrations by reading signals from enzyme reactions with a reader.

25. The microfluidic device of claim 2, wherein the specimen, cleaning solution, and substrate solution are provided in each microtubule pattern, the specimen and cleaning solution sequentially move through the microtubule patterns by capillary force to the above conjugate pad, the above reaction pad and an absorption pad without external power source, Wherein the cleaning solution move to an absorption pad, the substrate solution move to the space (A) under the reaction pad by capillary force through above microtubule patterns without external power sources.

26. The microfluidic device of claim 2, when the specimen reaches the conjugate pad, the microfluidic device moves the specimen to the reaction pad after the sensing antibodies or the aptamer in the conjugate pad make antigen-antibody reaction with the antigen (4) contained in the above specimen,
when specimen containing antigen-sensing antibody and unreacted sensing antibody reach the reaction pad, the microfluidic device above makes antigen-sensing antibody and unreacted sensing antibody react with antibody (51) and fix,
The microfluidic device above is a paper-based three-dimensional structure microfluidic device characterized by enzyme reaction between the substrate and the sensing material (31) combined with the sensing antibody when the substrate solution reaches the reaction pad.

27. The microfluidic device of claim 2, wherein the film area (B) between the first and second holes form a barrier layer that prevents above specimen and cleaning solution from leaking into above space (A).

28. The microfluidic device of claim 2, wherein the cover film (60) contains a hole (61) that reduces capillary force near the entrance of the reaction pad in which the specimen is introduced.

29. The microfluidic device of claim 2, wherein the paper is higher hydrophilicity compared to the films.

30. The microfluidic device of claim 2, wherein the specimen microtubule pattern part (213) and the cleaning solution microtubule pattern part are combined into one a pattern channel and then connected with the first hole.

31. The microfluidic device of claim 2, wherein the specimen microtubule pattern part (231) and the cleaning solution microtubule pattern part (232) are connected to the first hole.

32. The microfluidic device of claim 2, wherein the substrate solution reaches the 2nd hole later compared to the specimen solution or cleaning solution, since above substrate solution microtubule pattern has a wider or longer a pattern width than above specimen microtubule pattern part (231) and above cleaning solution a pattern part (232).
